SEQUENCE LISTING (22 sequences)

SEQ ID NO: 1 - DNA, 126 bp, Corynebacterium glutamicum
  misc_feature: seqP-RBS_01
ccagtcaatcctaagagcttgaaacgccccaatgtgggggtgttaagaactccataaaagcgcttgggaactttttgtggaagcagtccgttgaacctcttgaaccgcgaatttaggaggccagtt

SEQ ID NO: 2 - DNA, 151 bp, Corynebacterium glutamicum
  misc_feature: seqP-RBS_02
gacccctattatagcactgcttgataaattgtcaccggcccttagcctaagcttatttatattaggttgtcctatcctgaaagtgccgattcccgcaggccacgcttagttttttatcaaatcggcacaccggctactaaggttggtgatt

SEQ ID NO: 3 - DNA, 143 bp, Corynebacterium glutamicum
  misc_feature: seqP-RBS_03
tcttggatataaaaggggcccctattggcaatgtggttatgaccagaagtagtataagaagtgaagttgatcgcgtcatcgtagtacgagtaattccactcacacatcaatgcggtgaccacaattgggaggagaagtagcac

SEQ ID NO: 4 - DNA, 136 bp, Corynebacterium glutamicum
  misc_feature: seqP-RBS_04
gtcggtcgacctttcattgattggtgaatcctgcgttacagttcgtggaaactcatgatctctaacacctctaagtgtagcgcggatacccgatccgcacagctggttagtaccagtatttttgaggcttttgaca

SEQ ID NO: 5 - DNA, 639 bp, Corynebacterium glutamicum
  misc_feature: seqP-RBS_05
atgaagagaaataagccgacatttgatgtcggctttttcttttttctcgggctatttgagcttgagtcgggcggaaagcgcttcaacctgcggatttgtagaaaatctgggagtaacgtacatttaaacgcacaccagcaacgtggccaagaggttacaagctggttaagacatgcagaatgattaaaaaagaaactttgacttttttaaatcgactctgtaaggtcaatcaagctgctaggggagcaagtacaaacattgtatcttgttttcagagtgtgcgtatgttgtttgagaactcaatagtgtgccatttattatttttgtcactaccacaaccacttaggttgtggtgggtcatgccgggtggtggatcgccaattttatccatcactgtaaacaataatcttattgttggcatgattttgtgggtctgcttgttccccgtcaagggactaggcccacgttgaagacacgatgatccttatgtgggttgtggtgtttttttagaatcattattttttgataatgccagtaacaccccattatctgatcgcactgtgtgtggttggtgggggtgttgttgtttttgtcaggtgttgggcttttcacggcctgaacaatctatttattgttttt

SEQ ID NO: 6 - DNA, 489 bp, Corynebacterium glutamicum
  misc_feature: seqP-RBS_06
gttatcgaaaggtgatttttgcccagggccttgattcacaacgcaccttgctgtaggaaaaacaggcccctttgtgacatcggcgtagttgttcaactataatggaacgctgatcgtggacaagagttaaccatgagattgattcacccctttaagcctccaaagaagtagttgactcaacgcatttcggcatttaaaaaagccgagagcaaatgagactttccaggagaaggcaccagggacatgaacaattgatcggctgaccaactctataagagatgcacctcaagtttggggatacttattcggcgtttctggggaacaaatacgtttccctattgttgtatataggtattcgcacttaagaaacatctctcatggaaagaagctaggcggaaagggcgttaagtacttgccatttaatcctcagcatcactcggatcagtcggagatgtcgatgaaaatgcaccaggagccgtggagagcagc

SEQ ID NO: 7 - DNA, 1035 bp, Corynebacterium glutamicum
  misc_feature: seqP-RBS_07
tgcaccattcctctcttccttcgccggtgctaaccggacaggttcaaacggtgctcgcgcagcaatcagcgcgatctcagcccgtgctcgggcgcccgtggggactgccttcaaaactagcgcgttgcacgcgtgcgcgtaaaagttacccccttgcgggggttaagagagggcgcttttcgacgcggtgatggtggggtacgcctacgtagtggttctaactcggccgttgcttgcgaatctcagacacccgtgagtggtcttgaatccaattagtggcatctttctcaatccaagaggcgacattaaaaactgctgaatcaaacatattgaggttcctatcgtcgacgtctaaatgtgacttgctgagcggtgataattttcccaggagaatcgtttgttttagttcttcttttggcggttgttagagcttttttgaactgttgagcaaatctaaaatagctatcatcttcaaaccagaaggggccacgagcagatcgtaattgggcgtcaaccctgtttcttaaaagcacttctacatgttgtatatcttctaaatatgctttagataactgggtattccagatgtaaaaaccttcgggattttcagaatttcggtatgtattcattctagggatggagaaccactgttcgagctctgcgatagatgtaggggagttgccagtcacgtcgccaaggtcttacacttaggattcctaagccccggagtgatccctaccattttggtacgtcaccggggctcttgttttttcttcaagcgctcgtctctgatgtcctgaaagagtggctagggtatcggtgaatccatcgacacaaaaggattgggacattataaattcggcttgcatctagaccaagctgtctgctaatgtcgattttacgatcaagaagatcagccgcaagcccttcggcaggctgactggcaaccgcgcatcgcgcacggtgcccccgaaggaagatcggctctgtactcaaaagaatgcagagaagagcgattttttgaaggagattccc

SEQ ID NO: 8 - DNA, 134 bp, Corynebacterium glutamicum
  misc_feature: seqP-RBS_08
aatcttcatcacatggtgtgtgcagaaatatttttgctggtctattgtggcgaccgagggcctttgaaggttcgacaaactgtataaggccttgaatcttgagaatttattttgaggaagcaagaggaagtgtc

SEQ ID NO: 9 - DNA, 464 bp, Corynebacterium glutamicum
  misc_feature: seqP-RBS_09
taggtgcgctcttcccttctacaaatttttttgcttttcgacgccacaatgcagcgcgatttcagatcgccagcttaccccaaccgaagcccttcagaagacccgccccagcctatcaattttggtcaagtcgcctagctttagacctagctttagagcgcaatcccccaccccaaaacgtgatcttagtcacttattttccattacgccccacagggttcagtgtcagactaacgccacaagaccatctccatcagccctcaacttcaaccacttattgccctaaattatcgccactagcacgtcatttagagaaaacctttaatgtgaaacaggtctcaaactgggccattttcaagccttcaaaaaggtgggaaacttagccaatccaaagcccaaaaatgcgggttatgctgcgctaacctatgctgacagccttgcggaagttgtgtacgttaggggcc

SEQ ID NO: 10 - DNA, 245 bp, Corynebacterium glutamicum
  misc_feature: seqP-RBS_10
actaacttgatcaccttgctcggtgtctcgtaagcagcttgttcgactttacccttatcgttaatcagaaagcaacttgcgttaattcctgaagaattttgggagtctgtcagcgtttcaatttcccttgctgtgaagctcggttcgttgcgctgactcgaattaatctacacagcccctctgacaagcacaaacccccagctcaatgatggtttcgctgggggatgcttgatgggaaggcttcg

SEQ ID NO: 11 - DNA, 187 bp, Corynebacterium glutamicum
  misc_feature: seqP-RBS_11
aacttactttctcctagccgcccacattcagttagaagacgcgccgcaccgggtgaccggggcgcgagcatgccagctgacggggcgataacgaatatttacgacaacagcaagggacaagtttacatacgttgccccaaaacacctaatcgcctattcaatgcgggtttagatcgccccggaagcc

SEQ ID NO: 12 - DNA, 203 bp, Corynebacterium glutamicum
  misc_feature: seqP-RBS_12
aattgctcagaataccagttcagaccggggtcaccataaaggtgtgtagggggtgtcaaagtgggcaaaaagtatcatacttttgtctttttgctggcagtttacgtaagggctgattatttggtggcgcgaagtgacattctgatgtagactatcaatcagacttaatccataggtaaccctcataaaaggaaggaatgcta

SEQ ID NO: 13 - DNA, 124 bp, Corynebacterium glutamicum
  misc_feature: seqP-RBS_13
gcgttatagtctagagcgagcaggcgagatgtgaagtacctacacgcattaagtgcaaatgaattcacaattgccagaagatgcacaggatgtaatctagatttcccaagttcagtggggcaaa

SEQ ID NO: 14 - DNA, 205 bp, Corynebacterium glutamicum
  misc_feature: seqP-RBS_14
tgcagaatctcctccatcggcaggtacgtgccgaacgatccgtagtgtgagaaacagtgaagtgagtcactgtgcgccatctagagtgctgcctatgacattagcactagtttggaccctatgacaaagcccggaagccatgccgtggcttccgggcttagttcttctaattcatgggcgtggatgagacgccgcgggcatcgag

SEQ ID NO: 15 - DNA, 139 bp, Corynebacterium glutamicum
  misc_feature: seqP-RBS_15
cctgagaactatgaaattccagctcacggcgttttttcaggaaattctaggatcttacggagggtaactccgcactcaacatctagacaaaggctatgatgaaaatcgttaagaataatgtcgacacggaggaagttta

SEQ ID NO: 16 - DNA, 128 bp, Corynebacterium glutamicum
  misc_feature: seqP-RBS_16
ggacctcatcacctcctatggtctagtaattgttttttcggccatgccgtcgttggcatagcaggagggtcgttgcgtcagcaacctattcaaggtacaccaaacgcgcggaaatgtcgattcctgat

SEQ ID NO: 17 - DNA, 242 bp, Corynebacterium glutamicum
  misc_feature: seqP-RBS_17
cgctttagtctccttaactgttggccctttgaattacttttaggccgggacatcataggcttgcagtgtactcccctttttacggatctccggcgagtgatgctggattacgttcatatgggaagcggatgggtgttccccagcctactcaccgtccacagatgagtaaacccggaaaaacccgtatttagttattggttttacctgcgtgggctgaaagtcttcacttttaatccttacag

SEQ ID NO: 18 - DNA, 247 bp, Corynebacterium glutamicum
  misc_feature: seqP-RBS_18
ccccgatagtgtatgtgctgacctgcgtttatgcattttccttaggttgaaggaaatatcacacgacaaaagttgagtgatgcaggcataattggctataggcaactgaagatgccacaatcaatgtttgatccaacagaatcagacattgtgaatgtgggattatcggttcgcgcttcaccatgtttctgcatgatgaaattacatacatagttcagtgacagtcaccttttggaggagacacctt

SEQ ID NO: 19 - DNA, 205 bp, Corynebacterium glutamicum
  misc_feature: seqP-RBS_19
tggatacctgaaatcccagtgagcgcaccgctccccttacgtcacagtctgtaaaacaaatcttcggtgttgcgtatccttgttaataacttatgcgttgactcattcgtgcacttcggcgtgtcacaattaggtacgaccaagaatgggaccgggaaaccgggacgtataaacgaaataaaacattccaacaggaggtgtggaa

SEQ ID NO: 20 - DNA, 484 bp, Corynebacterium glutamicum
  misc_feature: seqP-RBS_20
ttgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaac

SEQ ID NO: 21 - DNA, 1263 bp, Corynebacterium glutamicum
  CDS (1)..(1263)
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcaccttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgc

SEQ ID NO: 22 - protein, 421 aa, Corynebacterium glutamicum
VALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

(19) United States
(12) Patent Application Publication
(10) Pub. No.: US 2008/0050786 A1
(43) Pub. Date: Feb. 28, 2008

(54) Method for producing L-amino acids

(75) Inventors: Brigitte Bathe, Salzkotten (DE); Stephan Hans, Osnabruck (DE); Wilfried Claes, Bielefeld (DE)

Correspondence Address: LAW OFFICE OF MICHAEL A. SANZO, LLC, 15400 CALHOUN DR., SUITE 125, ROCKVILLE, MD, 20855, US

(73) Assignee: Degussa GmbH, Dusseldorf (DE)

(21) Appl. No.: 11/822,454

(22) Filed: Jul. 6, 2007

Related U.S. Application Data
(60) Provisional application No. 60/831,243, filed on Jul. 17, 2006.

(30) Foreign Application Priority Data
Jul. 17, 2006 (EP) 06117294.6

(51) Int. Cl. C12P 13/08; C12N 1/15; C12N 1/20; C12P 13/12; C12P 13/06

(52) U.S. Cl. 435/113; 435/115; 435/116; 435/243; 435/252.3

(57) ABSTRACT

The invention is directed to a process for producing an L-amino acid by fermenting a microorganism, wherein the expression of a lysC gene encoding a feed back resistant aspartokinase is enhanced by cloning a strong promoter/ribosome binding sequence.

### CROSS REFERENCE TO RELATED APPLICATIONS

The present application claims priority to, and the benefit of, U.S. provisional application 60/831,243 filed on Jul. 17, 2006 and European application EP 06117294.6 filed on Jul. 17, 2006. The contents of these prior applications are hereby incorporated by reference in their entirety.

### FIELD OF THE INVENTION

The present invention is directed to methods of enhancing the fermentative production of amino acids by increasing the expression of a lysC gene encoding a feedback resistant aspartokinase. Enhanced expression is accomplished by cloning a polynucleotide encoding any of several specific promoter/ribosome binding site sequences in front of the lysC gene.

### BACKGROUND OF THE INVENTION

L-amino acids are used in the food sector, as pharmaceuticals and for animal nutrition. L-lysine is mainly used as additive for the preparation of animal feed. The current world production for L-lysine is estimated to be more than 350 thousand tons a year with production usually being carried out by the fermentation of Corynebacterium glutamicum. Review articles concerning various aspects of L-amino acids can be found in volume 79 of Advances in Biochemical Engineering Biotechnology (volume editors: R. Faurie and J. Thommel) entitled “Microbial Production of L-Amino Acids.”

The microbial L-lysine biosynthesis by Corynebacterium glutamicum is a well regulated process and many techniques have been used to increase production. For example, the copy number of a gene contributing to lysine biosynthesis may be increased or enzyme activity can be increased by raising the rate of gene expression. Recent patent applications demonstrate the use of various promoters for the overexpression of genes involved in L-amino acid biosyntheses or central metabolism (WO 2005/059144; WO 2005/059143; WO 2005/059093; WO 2006/008100; WO 2006/008101; WO 2006/008102; WO 2006/008103; WO 2006/008097; WO 2006/008098; US2006/0003424; WO 02/40679).

One enzyme of particular interest with respect to L-lysine biosynthesis is aspartokinase, an enzyme encoded by the lysC gene which catalyzes the phosphorylation of aspartate. This reaction is the first step in the biosynthesis of the “aspartate family” of essential amino acids, lysine, methionine and serine. Forms of aspartokinase that are resistant to feedback inhibition are known in the art and Corynebacteria with these enzymes have been reported to exhibit increased production of lysine (see e.g., U.S. Pat. No. 6,221,636). The ability to combine the benefits of an enzyme resistant to feedback inhibition with increased expression may lead to further improvements the fermentative production of amino acids

### SUMMARY OF THE INVENTION

The present invention is based upon the concept that the expression of a lysC gene encoding a feedback resistant aspartokinase can be enhanced by recombinantly incorporating one of the polynucleotides described below in table 1 in front of it. Each of the listed polynucleotides consists of a promoter sequence and a ribosome binding site. They can be found under the access number listed in Table 1 (column B) at the National Center for Biotechnology Information (NCBI, Bethesda, Md., USA). Under the specific accession number, the applied sequence spans from position 1 (column C of Table 1) to position 2 (column D of Table 1). The length of the polynucleotides is given in column E. The sequences comprising the applied polynucleotides are termed seqP-RBS_{—}01 to seqP-RBS_{—}20 (column F) and each has been given a corresponding sequence identification number (column G).

The polynucleotides comprising the promoter and ribosome binding site (seqP-RBS) and the coding sequence of the lysC gene may be cloned into plasmid pK18mobsacB (see US 20040043458, published on Mar. 4, 2004 and WO03/014330 for experimental details, incorporated herein by reference). The exchange of the natural lysC promoter contained in the chromosome of the parent strain against seqP-RBS is achieved by conjugation and subsequent screening for homologous recombination events as described in WO03/014330 and US 20040043458. Using these procedures, a bacterial strain is obtained which contains in its chromosome the seqP-RBS in front of the coding sequence of the lysC gene. Thus, the expression of aspartokinase is placed under the control of the seqP-RBS sequence.

Strains of Corynebacterium glutamicum carrying the combined seqP-RBS-lysC sequence produce significant higher amounts of L-lysine as compared to the parental strains. Enzyme activity analysis of these strains shows a higher amount aspartate kinase activity.

Other genes that may be overexpressed by placing the seqP-RBS sequences in front of their coding sequence in the manner described above include genes beneficial for L-lysine production such as those listed in WO03/014330 and US 20040043458, Table 1 (incorporated herein by reference). These include the following genes (accession numbers or references are shown in parentheses):

accBC (U35023; AX123524; AX066441)

accDA (AX121013;AX066443)

cstA (AX120811; AX066109)

cysD (AX123177)

cysE (AX122902; AX063961)

cysH (AX123178; AX066001)

cysK (AX122901; AX063963)

cysN (AX123176; AX127152)

cysQ (AX127145; AX066423)

dapA (X53993; Z21502; AX123560; AX063773)

dapB (AX127149; AX063753; AX137723; AX137602; X67737; Z21502; E16749; E14520; E12773; E08900)

dapC (AX127146; AX064219)

dapD (AX127146; AX63757; AJ004934)

dapE (AX127146; AX063749; X81379)

dapF (AX12749; AX063719; AX 137620)

ddh (AX127152; AX063759; Y00151; E14511; E05776; D87976)

dps (AX127153)

eno (AX127146; AX064945; AX136862)

gap (AX127148; AX064941; X59403)

gap2 (AX127146; AX064939)

gdh (AX127150; AX063811; X59404; X72855)

gnd (AX127147; AX121689; AX065125)

lysC (AX120365; AX063743; X57226)

lysE (AX123539; X96471)

msiK (AX120892)

opcA (AX076272)

oxyR (AX122198; AX127149)

ppcFBR (see EP072301 1; WO0100852)

ppc (AX127148; AX123554; M25819)

pgk (AX121838; AX127148; AX064943; X59403)

pknA (AX120131; AX120085)

pknB (AX120130; AX120085)

pknD (AX127150; AX122469; AX122468)

pknG (AX127152; AX123109)

ppsA (AX127144; AX120700; AX122469)

ptsH (AX122210; AX127149; AX069154)

ptsI (AX122206; AX127149)

pysM (L18874)

pyc and pyc (p458s) (A97276; Y09548)

sigC (AX120368; AX120085)

sigD (AX120753; AX127144)

sigE (AX127146; AX121325)

sigH (AX127145; AX120939

In particular expression of enzymes involved in the pentose phosphate pathway (e.g., the glucose 6 phosphate dehydrogenase complex encoded by genes zwf and opcA) or involved in anaplerosis (e.g. phosphoenolpyruvate carboxylase encoded by the ppc gene and pyruvate carboxylase encoded by the pyc gene) may be increased. In each case, the enhanced expression of these genes results in a significant increase in L-lysine production.

TABLE 1

| A | B | C | D | E | F | G |
|---|---|---|---|---|---|---|
| no. | access number | position 1 | position 2 | length (bp) | Name | SEQ ID NO: |
| 1 | BX927153.1 | 245335 | 245460 | 126 | seqP-RBS_01 | 1 |
| 2 | BX927157.1 | 32411 | 32561 | 151 | seqP-RBS_02 | 2 |
| 3 | BX927150.1 | 98920 | 99062 | 143 | seqP-RBS_03 | 3 |
| 4 | BX927156.1 | 323078 | 323213 | 136 | seqP-RBS_04 | 4 |
| 5 | BX927150.1 | 159092 | 159730 | 639 | seqP-RBS_05 | 5 |
| 6 | BX927153.1 | 235993 | 236481 | 489 | seqP-RBS_06 | 6 |
| 7 | BX927151.1 | 326822 | 327856 | 1035 | seqP-RBS_07 | 7 |
| 8 | BX927156.1 | 20664 | 20797 | 134 | seqP-RBS_08 | 8 |
| 9 | BX927155.1 | 201514 | 201977 | 464 | seqP-RBS_09 | 9 |
| 10 | BX927155.1 | 245359 | 245603 | 245 | seqP-RBS_10 | 10 |
| 11 | BX927150.1 | 228747 | 228933 | 187 | seqP-RBS_11 | 11 |
| 12 | BX927148.1 | 274121 | 274323 | 203 | seqP-RBS_12 | 12 |
| 13 | BX927151.1 | 120798 | 120921 | 124 | seqP-RBS_13 | 13 |
| 14 | BX927155.1 | 137815 | 138019 | 205 | seqP-RBS_14 | 14 |
| 15 | BX927152.1 | 125237 | 125375 | 139 | seqP-RBS_15 | 15 |
| 16 | BX927157.1 | 15642 | 15769 | 128 | seqP-RBS_16 | 16 |
| 17 | BX927154.1 | 207625 | 207866 | 242 | seqP-RBS_17 | 17 |
| 18 | BX927156.1 | 134993 | 135239 | 247 | seqP-RBS_18 | 18 |
| 19 | BX927154.1 | 256472 | 256676 | 205 | seqP-RBS_19 | 19 |
| 20 | BX927152.1 | 289885 | 289402 | 484 | seqP-RBS_20 | 20 |

### DETAILED DESCRIPTION OF THE INVENTION

Feedback resistant aspartokinase enzymes of C. glutamicum are described in U.S. Pat. No. 6,844,176 (incorporated herein by reference). They represent all proteins having the sequence shown herein as SEQ ID NO:22, but where one or more amino acid mutations have occurred resulting in feedback resistance. For example, the invention encompasses all sequences corresponding to SEQ ID NO:22 in which the serine at position 301 is replaced with a different proteinogenic L-amino acid, i.e., with an amino acid selected from: L-aspartic acid, L-asparagine, L-threonine, L-glutamic acid, L-glutamine, glycine, L-alanine, L-cysteine, L-valine, L-methionine, L-isoleucine, L-leucine, L-tyrosine, L-phenylalanine, L-histidine, L-lysine, L-tryptophan, L-proline and L-arginine. Feedback resistant lysC genes encode these enzymes. For example, a gene may have a nucleotide sequence corresponding to SEQ ID NO:21 but where the codon for the serine at position 301 in the enzyme has been changed. Other feedback resistant aspartokinase genes are described in EP 0387527; EP 0699759; and WO 00/63388. They are also described in published US application US 2004/0043458. All of these references are hereby incorporated by reference.

Examples of other feedback resistant forms of lysC (designated as lysC^{FBR}) that are included within the invention are: lysC^{FBR} A279T (replacement of alanine at position 279 of the aspartate kinase protein of SEQ ID NO:22 by threonine), lysC A279V (replacement of alanine at position 279 of the aspartate kinase protein coded by SEQ ID NO:22 by valine), lysc S301F (replacement of serine at position 301 of the aspartate kinase protein coded by SEQ ID NO:22 by phenylalanine), lysC T3081 (replacement of threonine at position 308 of the aspartate kinase protein coded by SEQ ID NO:22 by isoleucine), lysC S301Y (replacement of serine at position 308 of the aspartate kinase protein coded by SEQ ID NO:22 by tyrosine), lysc G345D (replacement of glycine at position 345 of the aspartate kinase protein coded by SEQ ID NO:22 by aspartic acid), lysc R320G (replacement of arginine at position 320 of the aspartate kinase protein coded by SEQ ID NO:22 by glycine), lysC T311I (replacement of threonine at position 311 of the aspartate kinase protein coded by SEQ ID NO:22 by isoleucine), lysc S381F (replacement of serine at position 381 of the aspartate kinase protein coded by SEQ ID NO:22 by phenylalanine).

The invention includes bacteria which are characterized by the enhanced expression of a feedback resistant aspartokinase encoded by a lysC^{FBR} gene. Overexpression is achieved by operably linking the lysC^{FBR} gene (see above) to one of the specific sequences listed in table 1. This is achieved by substituting the recombinant sequence for the promoter sequence normally associated with the gene by homologous recombination. As a result, the last nucleotide of the seqP-RBS will be within 800 nucleotides of the first nucleotide of the coding sequence and, more typically within 600, 400, or 200, 100 or. 50 nucleotides upstream of the first nucleotide of the coding region.

In another aspect, the invention is directed to a process for the fermentative production of an L-amino acid by: culturing the bacteria described above; allowing the fermentation medium or bacteria to become enriched in the amino acid; and then isolating it. It will be understood that the term L-amino acid as used herein refers to all biologically acceptable forms of these molecules, including all biologically acceptable salts.

The amino acids produced in the manner described above will most typically be used as part of, or to enrich, an animal feed. Usually, feeds and feed additives made by fermentation include some or all of the constituents of the fermentation medium and/or the biomass of bacteria. Thus, in most cases, these components will be isolated with the amino acid of interest. The most preferred amino acids to make by the process are those that are most directly subject to fluctuation based on lysC activity, L-lysine, L-methionine and L-serine, with L-lysine being most preferred. Particular preference is given to amino acid-secreting strains which are based on the following species:
Corynebacterium efficiens, for example the strain DSM44549,
Corynebacterium glutamicum, for example the strain ATCC13032,
Corynebacterium thermoaminogenes, for example the strain FERM BP-1539, and
Corynebacterium ammoniagenes, for example the strain ATCC6871

Corynebacterium glutamicum is particularly preferred. Some representatives of this species that may be used include, for example:
Corynebacterium acetoacidophilum ATCC 13870
Corynebacterium lilium DSM20137
Corynebacterium melassecola ATCC17965
Brevibacterium flavum ATCC14067
Brevibacterium lactofermentum ATCC13869 and
Brevibacterium divaricatum ATCC14020

Examples of known representatives of amino acid-secreting strains of coryneform bacteria preferred for the production of L-lysine are:
Corynebacterium glutamicum DM58-1/pDM6 (=DSM4697) described in EP 0 358 940
Corynebacterium glutamicum MH20 (=DSM5714) described in EP 0 435 132
Corynebacterium glutamicum AHP-3 (=FermBP-7382) described in EP 1 108 790
Corynebacterium thermoaminogenes AJ12521 (=FERM BP-3304) described in U.S. Pat. No. 5,250,423.

Information with regard to the taxonomic classification of strains of this group of bacteria can be found, inter alia, in Seiler (J. Gen. Microbiol. 129:1433-1477 (1983)), Kampfer, et al. (Can. J. Microbiol. 42:989-1005 (1996)), Liebl et al. (Int. J. Systematic Bacteriol. 41:255-260 (1991)) and in U.S. Pat. No. 5,250,434.

Strains having the designation “ATCC” can be obtained from the American Type Culture Collection (Manassas, Va., USA). Strains having the designation “DSM” can be obtained from the Deutsche Sammlung von Mikroorganismen und Zellkulturen [German Collection of Microorganisms and Cell Cultures] (DSMZ, Brunswick, Germany). Strains having the designation “FERM” can be obtained from the National Institute of Advanced Industrial Science and Technology (AIST Tsukuba Central 6, 1-1-1 Higashi, Tsukuba lbaraki, Japan). The abovementioned strains of Corynebacterium thermoaminogenes (FERM BP-1539, FERM BP-1540, FERM BP-1541 and FERM BP-1542) are described in U.S. Pat. No. 5,250,434.

“Proteinogenic amino acids” are understood as being the amino acids which occur in natural proteins, i.e., in proteins derived from microorganisms, plants, animals and humans. These amino acids include, in particular, L-amino acids selected from the group L-aspartic acid, L-asparagine, L-threonine, L-serine, L-glutamic acid, L-glutamine, glycine, L-alanine, L-cysteine, L-valine, L-methionine, L-isoleucine, L-leucine, L-tyrosine, L-phenylalanine, L-histidine, L-lysine, L-tryptophan, L-proline and L-arginine. The bacteria according to the invention preferably secrete the above-mentioned proteinogenic amino acids, in particular L-lysine. The terms “amino acids” or “L-amino acids” also encompass their salts such as lysine monohydrochloride or lysine sulfate.

Feedback-resistant aspartate kinases are understood as being aspartate kinases which exhibit less sensitivity, as compared with the wild form, to inhibition by mixtures of lysine and threonine, mixtures of AEC (aminoethylcysteine) and threonine, lysine on its own, or AEC on its own. The genes or alleles encoding these desensitized aspartate kinases are termed lysC^{FBR} alleles. A large number of lysC^{FBR} alleles which encode aspartate kinase variants, which possess amino acid substitutions as compared with the wild-type protein, are described in the prior art (see e.g., Table 2). The coding region of the wild-type lysC gene in Corynebacterium glutamicum corresponds to Accession Number AX756575 in the NCBI database.

TABLE 2
Feedback Resistant lysC Alleles

| | Amino Acid | | Access | |
|---|---|---|---|---|
| Allele Name | Replacement | Reference | Number | Allele No.^{a} |
| lysC^{FBR}-E05108 | | JP 1993184366-A | E05108 | 1 |
| | | (sequence 1) | | |
| lysC^{FBR}-E06825 | A279T | JP 1994062866-A | E06825 | 2 |
| | | (sequence 1) | | |
| lysC^{FBR}-E06826 | | JP 1994062866-A | E06826 | 3 |
| | | (sequence 2) | | |
| lysC^{FBR}-E06827 | | JP 1994062866-A | E06827 | 4 |
| | | (sequence 3) | | |
| lysC^{FBR}-E08177 | | JP 1994261766-A | E08177 | 5 |
| | | (sequence 1) | | |
| lysC^{FBR}-E08178 | A279T | JP 1994261766-A | E08178 | 6 |
| | | (sequence 2) | | |
| lysC^{FBR}-E08179 | A279V | JP 1994261766-A | E08179 | 7 |
| | | (sequence 3) | | |
| lysC^{FBR}-E08180 | S301F | JP 1994261766-A | E08180 | 8 |
| | | (sequence 4) | | |
| lysC^{FBR}-E08181 | T308I | JP 1994261766-A | E08181 | 9 |
| | | (sequence 5) | | |
| lysC^{FBR}-E08182 | | JP 1993184366-A | E08182 | 10 |
| lysC^{FBR}-E12770 | | JP 1997070291-A | E12770 | 11 |
| | | (sequence 13) | | |
| lysC^{FBR}-E14514 | | JP 1993322774-A | E14514 | 12 |
| | | (sequence 9) | | |
| lysC^{FBR}-E16352 | | JP 1998165180-A | E16352 | 13 |
| | | (sequence 3) | | |
| lysC^{FBR}-E16745 | | JP 1998215883-A | E16745 | 14 |
| | | (sequence 3) | | |
| lysC^{FBR}-E16746 | | JP 1998215883-A | E16746 | 15 |
| | | (sequence 4) | | |
| lysC^{FBR}-I74588 | | US 5688671 | I74588 | 16 |
| | | (sequence 1) | | |
| lysC^{FBR}-I74589 | A279T | US 5688671 | I74589 | 17 |
| | | (sequence 2) | | |
| lysC^{FBR}-I74590 | | US 5688671 | I74590 | 18 |
| | | (sequence 7) | | |
| lysC^{FBR}-I74591 | A279T | US 5688671 | I74591 | 19 |
| | | (sequence 8) | | |
| lysC^{FBR}-I74592 | | US5688671 | I74592 | 21 |
| | | (sequence 9) | | |
| lysC^{FBR}-I74593 | A279T | US5688671 | I74593 | 22 |
| | | (sequence 10) | | |
| lysC^{FBR}-I74594 | | US5688671 | I74594 | 23 |
| | | (sequence 11) | | |
| lysC^{FBR}-I74595 | A279T | US5688671 | I74595 | 24 |
| | | (sequence 12) | | |
| lysC^{FBR}-I74596 | | US5688671 | I74596 | 25 |
| | | (sequence 13) | | |
| lysC^{FBR}-I74597 | A279T | US5688671 | I74597 | 26 |
| | | (sequence 14) | | |
| lysC^{FBR}-X57226 | S301Y | EP0387527 | X57226 | 27 |
| | | Kalinowski, et al., Mol. | | |
| | | Gen. Genet. 224: 317-321 | | |
| | | (1990) | | |
| lysC^{FBR}-L16848 | G345D | Foilettie et al., NCBI | L16848 | 28 |
| | | Nucleotide database (1990) | | |
| lysC^{FBR}-L27125 | R3320G | Jetten, et al., Appl. | L27125 | 29 |
| | G345D | Microbiol.Biotechnol. | | |
| | | 43: 76-82 (1995) | | |
| lysC^{FBR} | T311I | WO0063388 | | 30 |
| | | (sequence 17) | | |
| lysC^{FBR} | S301F | US 3732144 | | 31 |
| lysC^{FBR} | S381F | | | 32 |
| lysC^{FBR} | | JP6261766 | | 33 |
| | | (sequence 1) | | |
| lysC^{FBR} | A279T | JP6261766 | | 34 |
| | | (sequence 2) | | |
| lysC^{FBR} | A279V | JP6261766 | | 35 |
| | | (sequence 3) | | |
| lysC^{FBR} | S301F | JP6261766 | | 36 |
| | | (sequence 4) | | |
| lysC^{FBR} | T308I | JP6261766 | | 37 |
| | | (sequence 5) | | |

(First 3 columns taken from US 2004/0043458. ^{a}Allele number for present application)

The activity of other enzymes that lead to an increase in bacterial amino acid production may also be enhanced by operably linking them to a sequence in table 1. The term “operably linked” indicates that the transcription of the lysC^{FBR} coding region is under the control of the promoter and ribosome binding site and that protein having the correct sequence is eventually produced. Especially preferred in this regard are genes involved in the pentose phosphate pathway. Endogenous genes associated with lysine production, other than lysC^{FBR} genes, that may be enhanced in this way include:
a dapA gene encoding a dihydropicolinate synthase, as, for example, the Corynebacterium glutamicum wild-type dapA gene described in EP 0 197 335,
a zwf gene encoding a glucose 6-phosphate dehydrogenase, as, for example, the Corynebacterium glutamicum wild-type zwf gene described in JP-A-09224661 and EP-A-1108790,
the Corynebacterium glutamicum zwf alleles which are described in US-2003-0175911-A1 and which encode a protein in which, for example, the L-alanine at position 243 in the amino acid sequence is replaced with L-threonine or in which the L-aspartic acid at position 245 is replaced with L-serine,
a pyc gene encoding a pyruvate carboxylase, as, for example, the Corynebacterium glutamicum wild-type pyc gene described in DE-A-198 31 609 and EP 1108790,
the Corynebacterium glutamicum pyc allele which is described in EP 1 108 790 and which encodes a protein in which L-proline at position 458 in the amino acid sequence is replaced with L-serine,
the Corynebacterium glutamicum pyc alleles which are modified as described in WO 02/31158;
a lysE gene which encodes a lysine export protein, as, for example, the Corynebacterium glutamicum wild-type lysE gene which is described in DE-A-195 48 222; and
the Corynebacterium glutamicum wild-type zwa1 gene encoding the Zwa1 protein (U.S. Pat. No. 6,632,644).

Further increases in bacterial amino acid production may also be achieved by attenuating one or more enzymes that limit production. In this connection, the term “attenuation” describes the reduction or elimination of the intracellular activity of one or more enzymes (proteins) which are encoded by the corresponding DNA This may be accomplished, for example, by using homologous recombination to substitute a weak promoter for a strong one or to disrupt to eliminate a gene.

The bacteria made in accordance with the invention can be cultured continuously, as described, for example, in PCT/EP2004/008882, or discontinuously, in a batch process or a fed-batch process or a repeated fed-batch process, for the purpose of producing L-amino acids. A general summary of known culturing methods can be found in the textbook by Chmiel (Bioprozesstechnik 1. Einführung in die Bioverfahrenstechnik [Bioprocess Technology 1. Introduction to Bioprocess Technology] (Gustav Fischer Verlag, Stuttgart, 1991)) or in the textbook by Storhas (Bioreaktoren und periphere Einrichtungen [Bioreactors and Peripheral Equipment] (Vieweg Verlag, Brunswick/Wiesbaden, 1994)).

The culture medium or fermentation medium to be used must satisfy the requirements of the given strains. Descriptions of media for culturing different microorganisms are given in the manual “Manual of Methods for General Bacteriolog” published by the American Society for Bacteriology (Washington D.C., USA, 1981). The terms culture medium and fermentation medium or medium are mutually interchangeable.

The carbon source employed can be sugars and carbohydrates, such as glucose, sucrose, lactose, fructose, maltose, molasses, sucrose-containing solutions derived from sugar beet or sugar cane production, starch, starch hydrolysate and cellulose, oils and fats, such as soybean oil, sunflower oil, peanut oil and coconut oil, fatty acids, such as palmitic acid, stearic acid and linoleic acid, alcohols, such as glycerol, methanol and ethanol, and organic acids, such as acetic acid. These substances can be used individually or as mixtures.

The nitrogen source employed can be organic nitrogen-containing compounds, such as peptones, yeast extract, meat extract, malt extract, cornsteep liquor, soybean flour and urea, or inorganic compounds, such as ammonium sulfate, ammonium chloride, ammonium phosphate, ammonium carbonate and ammonium nitrate. The nitrogen sources can be used individually or as mixtures.

The phosphorus source employed can be phosphoric acid, potassium dihydrogen phosphate or dipotassium hydrogen phosphate or the corresponding sodium-containing salts.

The culture medium must also contain salts, for example in the form of chlorides or sulfates of metals such as sodium, potassium, magnesium, calcium and iron, for example magnesium sulfate or iron sulfate, which are necessary for growth. Finally, essential growth substances, such as amino acids, for example homoserine, and vitamins, for example thiamine, biotin or pantothenic acid, can be used in addition to the above-mentioned substances. In addition to this, suitable precursors of amino acids can be added to the culture medium.

The above-mentioned substances can be added to the culture in the form of a once-only mixture or fed in a suitable manner during the culture.

Basic compounds such as sodium hydroxide, potassium hydroxide, ammonia or ammonia water, or acidic compounds such as phosphoric acid or sulfuric acid, are employed in a suitable manner for controlling the pH of the culture. In general, the pH is adjusted to a value of from 6.0 to 9.0, preferably of from 6.5 to 8. It is possible to use antifoamants, such as fatty acid polyglycol esters, for controlling foam formation. Suitable substances which act selectively, such as antibiotics, can be added to the medium in order to maintain the stability of plasmids. In order to maintain aerobic conditions, oxygen or oxygen-containing gas mixtures, such as air, are passed into the culture. It is also possible to use liquids which are enriched with hydrogen peroxide. Where appropriate, the fermentation is conducted under positive pressure, for example under a pressure of 0.03 to 0.2 MPa. The temperature of the culture is normally from 20° C. to 45° C., and preferably from 25° C. to 40° C. In the case of batch processes, the culture is continued until a maximum of the desired amino acid has been formed. This objective is normally achieved within from 10 hours to 160 hours. Longer culturing times are possible in the case of continuous processes.

Suitable fermentation media are described, inter alia, in U.S. Pat. No. 6,221,635, U.S. Pat. No. 5,840,551, U.S. Pat. No. 5,770,409, U.S. Pat. No. 5,605,818, U.S. Pat. No. 5,275,940 and U.S. Pat. No. 4,224,409.

At the conclusion of the fermentation, the resulting fermentation broth contains a) the biomass of the microorganism which has been formed as a consequence of the replication of the cells of the microorganism, b) the desired amino acid which has been formed during the fermentation, c) the organic by-products which have been formed during the fermentation, and d) the constituents of the fermentation medium employed, or the added substances, for example vitamins, such as biotin, amino acids, such as homoserine, or salts, such as magnesium sulfate, which were not consumed by the fermentation.

The organic by-products include substances which are produced by the microorganisms employed in the fermentation in addition to the given desired L-amino acid. These by-products include L-amino acids which amount to less than 30%, 20% or 10% of the desired amino acid. They also include organic acids which carry from 1 to 3 carboxyl groups, such as acetic acid, lactic acid, citric acid, malic acid or fumaric acid. Finally, they also include sugars, such as trehalose.

Methods for determining L-amino acids are disclosed in the prior art. The analysis can, for example, take place by means of anion exchange chromatography, followed by ninhydrin derivatization, as described in Spackman et al. (Anal. Chem. 30:1190 (1958)), or it can take place by reversed phase HPLC, as described in Lindroth, et al. (Anal. Chem. 51:1167-1174 (1979)). Typical fermentation broths which are suitable for industrial purposes have an amino acid content of from 40 g/kg to 180 g/kg or of from 50 g/kg to 150 g/kg. In general, the content of biomass (as dry biomass) is from 20 to 50 g/kg.

In the case of the amino acid L-lysine, essentially four different product forms have been disclosed in the prior art. One group of L-lysine-containing products comprises concentrated, aqueous, alkaline solutions of purified L-lysine (EP-B-0534865). Another group, as described, for example, in U.S. Pat. No. 6,340,486 and U.S. Pat. No. 6,465,025, comprises aqueous, acidic, biomass-containing concentrates of L-lysine-containing fermentation broths. The most well known group of solid products comprises pulverulent or crystalline forms of purified or pure L-lysine, which is typically present in the form of a salt such as L-lysine monohydrochloride. Another group of solid product forms is described, for example, in EP-B-0533039. The product form which is described in this document typically contains, in addition to L-lysine, the major portion of the added substances which were used during the fermentative preparation, and which were not consumed, and, where appropriate, from >0% to 100% of the biomass of the microorganism employed.

In correspondence with the different product forms, a very wide variety of methods are known for collecting or purifying the L-amino acid from the fermentation broth for the purpose of preparing the L-amino acid-containing product or the purified L-amino acid. It is essentially ion exchange chromatography methods, where appropriate using active charcoal, and crystallization methods which are used for preparing solid, pure L-amino acids. In the case of lysine, this results in the corresponding base or a corresponding salt such as the monohydrochloride (Lys-HCl) or the lysine sulfate (Lys₂-H₂SO₄).

As far as lysine is concerned, EP-B-0534865 describes a method for preparing aqueous, basic L-lysine-containing solutions from fermentation broth. In this document, the biomass is separated off from the fermentation broth and discarded. A base such as sodium hydroxide, potassium hydroxide or ammonium hydroxide is used to adjust the pH to between 9 and 11. Following concentration and cooling, the mineral constituents (inorganic salts) are separated off from the broth by crystallization and either used as fertilizer or discarded.

Depending on the intended use of the product, the biomass can be entirely or partially removed from the fermentation broth by means of separation methods such as centrifugation, filtration or decanting, or a combination of these methods, or all the biomass can be left in the fermentation broth. Where appropriate, the biomass, or the biomass-containing fermentation broth, is inactivated during a suitable process step, for example by means of thermal treatment (heating) or by means of adding acid.

In one approach, the biomass is completely or virtually completely removed, such that no (0%) or at most 30%, at most 20%, at most 10%, at most 5%, at most 1% or at most 0.1%, of the biomass remains in the prepared product. In another approach, the biomass is not removed, or only removed in trivial amounts, such that all (100%) or more than 70%, 80%, 90%, 95%, 99% or 99.9% of the biomass remains in the prepared product. In one process according to the invention, the biomass is accordingly removed in proportions of from ≧0% to <100%.

Finally, the fermentation broth which is obtained after the fermentation can be adjusted, before or after the biomass has been completely or partially removed, to an acid pH using an inorganic acid, such as hydrochloric acid, sulfuric acid or phosphoric acid, or an organic acid, such as propionic acid (GB 1,439,728 or EP 1 331 220). It is likewise possible to acidify the fermentation broth when it contains the entire biomass. Finally, the broth can also be stabilized by adding sodium bisulfite (NaHSO₃, GB 1,439,728) or another salt, for example an ammonium, alkali metal or alkaline earth metal salt of sulfurous acid.

Organic or inorganic solids which may be present in the fermentation broth are partially or entirely removed when the biomass is separated off. At least some (>0%), preferably at least 25%, particularly preferably at least 50%, and very particularly preferably at least 75%, of the organic by-products which are dissolved in the fermentation broth and the constituents of the fermentation medium (added substances), which are dissolved and not consumed remain in the product. Where appropriate, these by-products and constituents also remain completely (100%) or virtually completely, that is >95% or >98%, in the product. In this sense, the term “fermentation broth basis” means that a product comprises at least a part of the constituents of the fermentation broth.

Subsequently, water is extracted from the broth, or the broth is thickened or concentrated, using known methods, for example using a rotary evaporator, a thin-film evaporator or a falling-film evaporator, or by means of reverse osmosis or nanofiltration. This concentrated fermentation broth can then be worked up into flowable products, in particular into a finely divided powder or, preferably, a coarse-grained granulate, using methods of freeze drying, of spray drying or of spray granulation, or using other methods, for example in a circulating fluidized bed as described in PCT/EP2004/006655. Where appropriate, a desired product is isolated from the resulting granulate by means of screening or dust separation. It is likewise possible to dry the fermentation broth directly, i.e., by spray drying or spray granulation without any prior concentration.

The flowable, finely divided powder can in turn be converted, by means of suitable compacting or granulating methods, into a coarse-grained, readily flowable, storable, and to a large extent dust-free, product. The term “dust-free” means that the product only contains small proportions (<5%) of particle sizes of less than 100 μm in diameter. Within the meaning of this invention, “storable” means a product which can be stored for at least one (1) year or longer, preferably at least 1.5 years or longer, particularly preferably two (2) years or longer, in a dry and cool environment without there being any significant loss (<5%) of the given amino acid.

It is advantageous to use customary organic or inorganic auxiliary substances, or carrier substances such as starch, gelatin, cellulose derivatives or similar substances, as are customarily used as binders, gelatinizers or thickeners in foodstuff or feedstuff processing, or other substances, such as silicic acids, silicates (EP0743016A) or stearates, in connection with the granulation or compacting. It is also advantageous to provide the surface of the resulting granulates with oils, as described in WO 04/054381. The oils which can be used are mineral oils, vegetable oils or mixtures of vegetable oils. Examples of these oils are soybean oil, olive oil and soybean oil/lecithin mixtures. In the same way, silicone oils, polyethylene glycols or hydroxyethyl cellulose are also suitable. Treating the surfaces with these oils increases the abrasion resistance of the product and reduces the dust content. The content of oil in the product is from 0.02 to 2.0% by weight, preferably from 0.02 to 1.0% by weight, and very particularly preferably from 0.2 to 1.0% by weight, based on the total quantity of the feedstuff additives.

Preference is given to products having a content of >97% by weight of a particle size of from 100 to 1800 μm, or a content of >95% by weight of a particle size of from 300 to 1800 μm, in diameter. The content of dust, ie., particles having a particle size of <100 μm, is preferably from >0 to 1% by weight, particularly preferably at most 0.5% by weight.

Alternatively, the product can also be absorbed onto an organic or inorganic carrier substance which is known and customary in feedstuff processing, for example silicic acids, silicates, grists, brans, meals, starches, sugars etc., and/or be mixed and stabilized with customary thickeners or binders. Application examples and methods in this regard are described in the literature (Die Mühle+Mischfuttertechnik [The Grinding Mill+Mixed Feed Technology] 132 (1995) 49, page 817).

Finally, the product can be brought, by means of coating methods using film formers such as metal carbonates, silicic acids, silicates, alginates, stearates, starches, rubbers and cellulose ethers, as described in DE-C-4100920, into a state in which it is stable towards digestion by animal stomachs, in particular the ruminant stomach.

In order to set a desired amino acid concentration in the product, the appropriate amino acid can, depending on the requirement, be added during the process in the form of a concentrate or, where appropriate, of a largely pure substance or its salt in liquid or solid form. The latter can be added individually, or as mixtures, to the resulting fermentation broth, or to the concentrated fermentation broth, or else added during the drying process or granulation process.

In the case of lysine, the ratio of the ions may be adjusted during the preparation of lysine-containing products such that the ion ratio in accordance with the following formula:
2×[SO₄²⁻]+[Cl⁻]—[NH₄⁺]—[Na⁺]—[K⁺]—2×[Mg⁺]-2×[Ca²⁺]/[L-Lys]
has a value of from 0.68 to 0.95, preferably of from 0.68 to 0.90, as described by Kushiki et al. in US 20030152633.

In the case of lysine, the solid fermentation broth-based product which has been prepared in this way may have a lysine content (as lysine base) of from 10% by weight to 70% by weight or of from 20% by weight to 70% by weight, preferably of from 30% by weight to 70% by weight and very particularly preferably of from 40% by weight to 70% by weight, based on the dry mass of the product. It is also possible to achieve maximum contents of lysine base of 71% by weight, 72% by weight or 73% by weight. The water content of the solid product may be up to 5% by weight, preferably up to 4% by weight, and particularly preferably less than 3% by weight.

### EXAMPLES

A polynucleotide comprising the promoter/RBS sequence of any one of SEQ ID NO:1-SEQ ID NO:20 is cloned into plasmid pK18mobsacB (see WO03/014330 and US 20040043458 for experimental details). The exchange of the natural lysC promoter contained in the chromosome of the parent strain is achieved by conjugation and subsequent screening for homologous recombination events as described. A strain is obtained which contains in its chromosome the promoter/RBS sequence in front of the coding sequence of the lysC^{FBR} gene.

Strains of Corynebacterium glutamicum carrying the promoter/RBS sequence produce significantly higher amounts of L-lysine as compared to the parental strains. Enzyme activity analysis of these strains shows a higher aspartate kinase activity.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. An isolated microorganism comprising within its genome a nucleotide sequence encoding a feedback resistant aspartokinase enzyme, operably linked to a sequence selected from the group consisting of SEQ ID NO:1 -SEQ ID NO:20.

2. The isolated microorganism of claim 1, wherein said feedback resistant aspartokinase enzyme comprises the amino acid sequence of SEQ ID NO:22 except that a proteinogenic L-amino acid other than L-serine is present at position 301.

3. The isolated microorganism of claim 1, wherein said microorganism is produced by homologous recombination.

4. The isolated microorganism of claim 2, wherein said microorganism is a coryneform bacterium.

5. The isolated microorganism of claim 4, wherein said coryneform bacterium is C. glutamicum.

6. A process for the fermentative production of an L-amino acid comprising:
a) culturing the coryneform bacterium of claim 1 in a fermentation medium;
b) allowing said fermentation medium or said coryneform bacterium to become enriched in said L-amino acid; and
c) isolating said L-amino acid.

7. The process of claim 6, wherein some or all of the constituents of said fermentation medium and/or the biomass of said coryneform bacterium are isolated with said L-amino acid.

8. The process of claim 6, wherein said L-amino acid is selected from the group consisting of L-lysine, L-methionine and L-serine.

9. The process of claim 6, wherein said L-amino acid is L-lysine.

10. The process of claim 9, wherein said coryneform bacterium is C. glutamicum.
